(19) United States
(12) Patent Application Publication
(10) Pub. No.: US 2008/0187582 A1
(43) Pub. Date: Aug. 7, 2008

(54) Pharmaceutical Composition Comprising an Indolylmaleimide Derivative

(76) Inventors: Patrice Guitard, Hegenheim (FR); Marie-Christine Wolf, St. Louis (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/815,865

(22) PCT Filed: Feb. 27, 2006
(86) PCT No.: PCT/EP2006/001767

§ 371 Date: Sep. 20, 2007

(30) Foreign Application Priority Data
Mar. 1, 2005 (GB) 0504203.1

(51) Int. Cl. A61K 9/20; A61K 31/404; A61K 31/517; A61K 31/4725

(52) U.S. Cl. 424/464; 514/414; 514/266.23; 514/278

(57) ABSTRACT

The application relates to solid pharmaceutical compositions suitable for oral administration comprising a water sensitive drug, preferably an indolylmaleimide derivative, process for their production and use of the pharmaceutical compositions.

The present invention relates to solid pharmaceutical compositions suitable for oral administration comprising a water sensitive drug, preferably an indolylmaleimide derivative, process for their production and use of the pharmaceutical compositions.

Pharmaceutical compositions for oral administration in a solid form are in general known as well as methods of producing the same. For example, a tablet form may be produced by dry compression, e.g. direct compression or roller compaction. However, there is a need for a water sensitive drug, preferably an indolylmaleimide derivative, containing solid pharmaceutical composition which is adapted for oral administration, preferably in the form of a tablet. Also, there is a need for said compositions having a high drug load, e.g. a drug load greater than 20%.

Accordingly, the present invention provides a solid pharmaceutical composition suitable for oral administration comprising a water sensitive drug, preferably an indolylmaleimide derivative, suitable to achieve high drug loads.

It has now surprisingly been found a solid pharmaceutical composition comprising a water sensitive drug, preferably an indolylmaleimide derivative, suitable for oral administration. The composition according to the present invention may, in addition, show a high level of uniformity in the distribution of the drug as well as high stability. The composition according to the present invention may be manufactured on high speed automated equipment, avoiding time-consuming encapsulation techniques.

By water sensitive drug is meant an active agent which is highly soluble in water and in ethanol with a high powder-liquid ratio, e.g. a ratio of 10 mg/ml, and which may convert either to a free base hydrate, a solvate or an amorphous form in the presence of ethanol and/or water.

More particularly the present invention relates to a solid pharmaceutical composition suitable for oral administration containing an indolylmaleimide derivative of formula X

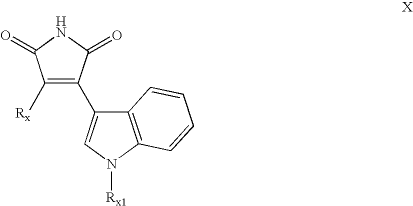

wherein Rₓ is an aromatic or heterocyclic residue, e.g. as defined below, and Rₓ₁ is H or a substituent, e.g. as indicated below, the indolyl residue being optionally further substituted, e.g. by one or 2 substituents.

Representative indolylmaleimide derivatives are e.g. compounds of formula I

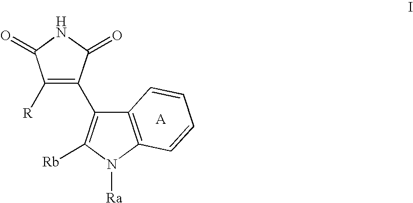

wherein
Rₐ is H; C₁₋₄alkyl; or C₁₋₄alkyl substituted by OH, NH₂, NHC₁₋₄alkyl or N(di-C₁₋₄alkyl)₂;
R_{b} is H; or C₁₋₄alkyl;
R is a radical of formula (a), (b), (c), (d), (e) or (f)

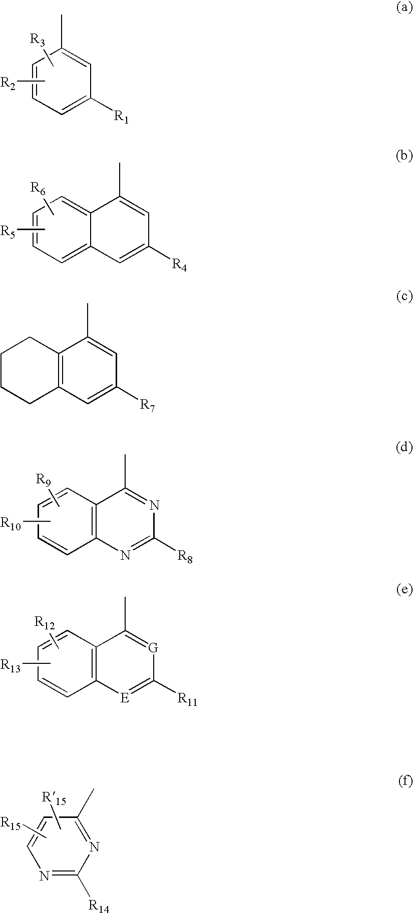

wherein
each of R₁, R₄, R₇, R₈, R₁₁ and R₁₄ is OH; SH; a heterocyclic residue; NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α

—X—R_{c}—Y (α)
wherein X is a direct bond, O, S or NR₁₈ wherein R₁₈ is H or C₁₋₄alkyl,
R_{c} is C₁₋₄alkylene or C₁₋₄alkylene wherein one CH₂ is replaced by CRₓR_{y} wherein one of Rₓ and R_{y} is H and the other is CH₃, each of Rₓ and R_{y} is CH₃ or Rₓ and R_{y} form together —CH₂—CH₂—, and
Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₉ and R₂₀ form together with the nitrogen atom to which they are bound a heterocyclic residue;
each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;
either E is —N═ and G is —CH═ or E is —CH═ and G is —N═; and
ring A is optionally substituted.

Any alkyl or alkyl moiety in e.g. alkoxy may be linear or branched. Halogen may be F, Cl, Br or I, preferably F or Cl. Any aryl may be phenyl or naphthyl, preferably phenyl.

By heterocyclic residue as R₁, R₄, R₇, R₈, R₁₁, R₁₄ or Y or formed, respectively, by NR₁₆R₁₇ or NR₁₉R₂₀, is meant a three to eight, preferably five to eight, membered saturated, unsaturated or aromatic heterocyclic ring comprising 1 or 2 heteroatoms, preferably selected from N, O and S, and optionally substituted. Suitable examples include e.g. pyridyl, e.g. 3- or 4-pyridyl, piperidyl, e.g. piperidin-1-yl, 3- or 4-piperidyl, homopiperidyl, piperazinyl, homopiperazinyl, morpholin-4-yl, imidazolyl, imidazolidinyl, pyrrolyl or pyrrolidinyl, optionally substituted, e.g. mono- or polysubstituted. When the heterocyclic residue is substituted, this may be on one or more ring carbon atoms and/or on a ring nitrogen atom when present. Examples of a substituent on a ring carbon atom include e.g. C₁₋₄alkyl e.g. CH₃;

C₃₋₆cycloalkyl e.g. cyclopropyl, optionally further substituted by C₁₋₄alkyl;

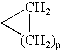

wherein p is 1, 2 or 3, preferably 1; CF₃; halogen; OH; NH₂; —CH₂—NH₂; —CH₂—OH; piperidin-1-yl; or pyrrolidinyl. Examples of a substituent on a ring nitrogen atom are e.g. C₁₋₆alkyl; acyl, e.g. R′ₓ—CO wherein R′ₓ is H, C₁₋₆alkyl or phenyl optionally substituted by C₁₋₄alkyl, C₁₋₄alkoxy or amino, e.g formyl; C₃₋₆cycloalkyl; C₃₋₆cycloalkyl-C₁₋₄alkyl; phenyl; phenyl-C₁₋₄alkyl e.g. benzyl; a heterocyclic residue, e.g. as disclosed above, e.g. an aromatic heterocyclic residue comprising 1 or 2 nitrogen atoms; or a residue of formula β,

—R₂₁—Y′ (β)

wherein R₂₁ is C₁₋₄alkylene or C₂₋₄alkylene interrupted by O and Y′ is OH, NH₂, NH(C₁₋₄alkyl) or N(C₁₋₄alkyl)₂.

C₂₋₄alkylene interrupted by O may be e.g. —CH₂—CH₂—O—CH₂—CH₂—.

When the substituent on a cyclic nitrogen is a heterocyclic residue, it may be a five or six membered saturated, unsaturated or aromatic heterocyclic ring comprising 1 or 2 heteroatoms, preferably selected from N, O and S. Examples include e.g. 3- or 4-pyridyl, piperidyl, e.g. piperidin-1-yl, 3- or 4-piperidyl, homopiperidyl, piperazinyl, homopiperazinyl, pyrimidinyl, morpholin-4-yl, imidazolyl, imidazolidinyl, pyrrolyl or pyrrolidinyl,

When Rₐ is substituted C₁₋₄alkyl, the substituent is preferably on the terminal carbon atom.

When ring A is substituted, it may be mono- or polysubstituted, preferably monosubstituted, the substituent(s) being selected from the group consisting of e.g. halogen, OH, C₁₋₄alkoxy, e.g. OCH₃, C₁₋₄alkyl, e.g. CH₃, NO₂, CF₃, NH₂, NHC₁₋₄alkyl, N(di-C₁₋₄alkyl)₂ and CN. For example, ring A may be a residue of formula

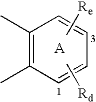

wherein
R_{d} is H; C₁₋₄alkyl; or halogen; and
Rₑ is OH; NO₂; NH₂; NHC₁₋₄alkyl; or N(di-C₁₋₄alkyl)₂.

Preferably R_{d} is in position 1; preferably Rₑ is in position 3.

When R_{c} has a CH₂ replaced by CRₓR_{y}, it is preferably the CH₂ bearing Y.

Examples of heterocyclic residue as R₁, R₄, R₇, R₈, R₁₁, R₁₄ or Y or formed, respectively, by NR₁₆R₁₇ or NR₁₉R₂₀, include e.g. a residue of formula (γ)

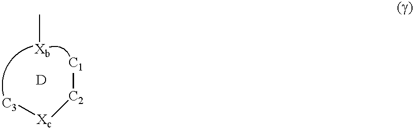

wherein
the ring D is a 5, 6 or 7 membered saturated, unsaturated or aromatic ring;
X_{b} is —N—, —C═ or —CH—;
X_{c} is —N═, —NH, —NR_{f}—, —CR′_{f}═ or —CHR′_{f}— wherein R_{f} is a substituent as indicated above for a ring nitrogen atom, and R′_{f} is a substituent as indicated above for a ring carbon atom;
the bond between C₁ and C₂ is either saturated or unsaturated;
each of C₁ and C₂, independently, is a carbon atom which is optionally substituted by one or two substituents selected among those indicated above for a ring carbon atom; and
the line between C₃ and X_{b} and between C₁ and X_{b}, respectively, represents the number of carbon atoms as required to obtain a 5, 6 or 7 membered ring D.

A preferred residue of formula (γ) is one wherein the ring D forms a 1,4-piperazinyl ring optionally C- and/or N-substituted as indicated.

Representative examples of a residue of formula (γ) are e.g. 3- or 4-pyridyl; piperidin-1-yl; 1-N—(C₁₋₄alkyl)- or -(ω-hydroxy-C₁₋₄alkyl)-3-piperidyl; morpholin-4-yl; imidazolyl; pyrrolidinyl; 1-piperazinyl; 2-C₁₋₄alkyl- or —C₃₋₆cycloalkyl-1-piperazinyl; 3-C₁₋₄alkyl- or —C₃₋₆cycloalkyl-1-piperazinyl; 2,2- or 3,5- or 2,5- or 2,6-di(C₁₋₄alkyl)-1-piperazinyl; 3,4,5-tri-(C₁₋₄alkyl)-1-piperazinyl; 4-N—(C₁₋₄alkyl)- or -(co-hydroxy-C₁₋₄alkyl)- or -(ω-dimethylamino-C₁₋₄alkyl)-1-piperazinyl; 4-N-pyridin-4-yl-1-piperazinyl; 4-N-phenyl- or —C₃₋₆cycloalkyl-1-piperazinyl; 4-N—(C₁₋₄alkyl)- or -(ω-hydroxy-C₁₋₄alkyl)-3-C₁₋₄alkyl- or -3,3-di(C₁₋₄alkyl)-1-piperazinyl; 4-N-(1-C₁₋₄alkyl-C₃₋₆cycloalkyl)-1-piperazinyl; 4-N-formyl-1-piperazinyl; 4-N-pyrimidin-2-yl-1-piperazinyl; 4-N—C₁₋₄alkyl-1-homopiperazinyl; or 4,7-diaza-spiro[2.5]oct-7-yl.

The compounds of formula I may exist in free form or in salt form, e.g. addition salts with e.g. organic or inorganic acids, for example, hydrochloric acid, acetic acid, when R₁, R₄, R₇, R₈, R₁₁ or R₁₄ and/or R₂, R₃, R₅, R₆, R₉, R₁₀, R₁₂, R₁₃ or R₁₅ comprises an optionally substituted amino group or a heterocyclic residue which can form acid addition salts.

It will be appreciated that the compounds of formula I may exist in the form of optical isomers, racemates or diastereoisomers. For example, a ring carbon atom bearing a substituent in the heterocyclic residue as R₁, R₄, R₇, R₈, R₁₁, R₁₄ or Y or formed, respectively, by NR₁₆R₁₇ or NR₁₉R₂₀, is asymmetric and may have the D- or L-configuration. It is to be understood that the present invention embraces all enantiomers and their mixtures. Similar considerations apply in relation to starting materials exhibiting assymetric carbon atoms as mentioned.

In the compounds of formula I, the following significances are preferred individually or in any sub-combination:
1. Rₐ is H or CH₃;
2. R_{b} is H;
3. Ring A is unsubstituted; or is substituted by methyl in position 7;
4. Preferred heterocyclic residue as formed by NR₁₆R₁₇ is e.g. piperazin-1-yl optionally N-substituted, e.g. by C₁₋₄alkyl, ω-hydroxy-C₁₋₄alkyl, ω-dimethylamino-C₁₋₄alkyl, C₅₋₆cycloalkyl, C₁₋₄alkyl-C₅₋₆cycloalkyl, an aromatic heterocyclic residue comprising 1 or 2 nitrogen atoms, e.g. pyridyl or pyrimidin-2-yl or 4,7-diaza-spiro[2.5]oct-7-yl; or a residue of formula β as defined above and/or optionally C-substituted, e.g. by CH₃ e.g. in positions 2, and/or 3 and/or 5 and/or 6 and/or 2,2 or 3,3 or by

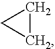

e.g. in position 2 or 3; piperidin-1-yl optionally C-substituted, e.g. in position 4, by NH₂, —CH₂—NH₂ or piperidin-1-yl, or in position 3, e.g. by OH or NH₂; or pyrrolidinyl optionally C-substituted in position 3 by OH or NH₂;
5. R₁₈ is H or CH₃;
6. R_{c} is C₁₋₄alkylene or C₁₋₄alkylene wherein the terminal CH₂ is replaced by CRₓ, R_{y} wherein Rₓ and R_{y} form together —CH₂—CH₂—;
7. X is O;
8. The radical of formula (α) is —O—CH₂—CH₂—Y;
9. Each of R₁₉ and R₂₀ is H, C₁₋₄alkyl, e.g. methyl, C₁₋₄alkyl substituted on the terminal carbon atom by OH, e.g. —CH₂—CH₂—OH, or cyclopropyl;
10. Preferred heterocyclic residue as formed by NR₁₉R₂₀ is e.g. piperazin-1-yl optionally N-substituted by C₁₋₄alkyl or a residue of formula β; piperidin-1-yl; 1-(C₁₋₄alkyl)-piperidin-3-yl; 3- or 4-pyridyl; imidazolyl; pyrrolidinyl; or morpholin-4-yl;
11. Each of R₁, R₄, R₇, R₈, R₁₁ or R₁₄, independently, is 1-N-methyl-piperidin-4-yl; 4-methyl-piperazin-1-yl; 4-methyl-1-homopiperazinyl; 4-(2-hydroxyethyl)-piperazin-1-yl; or —X′—C_{1, 2 or 3}-alkylene-NR₁₉R₂₀ wherein X′ is a direct bond, O or NH;
12. In the residue of formula (a) either each of R₂ and R₃ is H or one of R₂ and R₃ is H and the other is F, Cl, CH₃, OH, OCH₃ or CF₃;
13. In the residue of formula (a) R₂ is OH;
14. In the residue of formula (b) either each of R₅ and R₆ is H or one of R₅ and R₆ is H and the other is F, Cl, CH₃, OCH₃ or CF₃;
15. In the residue of formula (b) R₄ is a radical of formula (α) or NR₁₆R₁₇;
16. In the residue of formula (d) either each of R₉ and R₁₀ is H or one of R₉ and R₁₀ is H and the other is F, Cl, CH₃, OCH₃ or CF₃; preferably R₁₀ is H and R₉ is in position 5, 6, 7 or 8, preferably in position 6;
17. In the residue of formula (e) each of R₁₂ and R₁₃ is H;
18. In the residue of formula (e) one of R₁₂ and R₁₃ is H and the other is F, Cl, CH₃, OCH₃ or CF₃;
   when E is —N═ and G is —CH═, preferably R₁₃ is H and R₁₂ is in position 6 or 7;
   when E is —CH═ and G is —N═, preferably R₁₃ is H and R₁₂ is in position 7;
19. In the residue of formula (f) R₁₅ is H, CH₃ or Cl, e.g. in position 5 or 6;
20. In the residue of formula (f) R′₁₅ is H or CH₃, e.g. in position 5, preferably H;
21. R is a radical of formula (d), (e) or (f).

Compounds of formula I and their preparation are known, e.g. they are disclosed in WO02/38561 and WO03/82859, the contents thereof being incorporated herein by reference.

According to the invention, there is provided a solid pharmaceutical composition suitable for oral administration comprising from 20 to 70%, preferably 20 to 55% by weight of a water sensitive drug, preferably an indolylmaleimide derivative and most preferred a compound of formula I in free form or in pharmaceutically acceptable salt, preferably from 15 to 80%, preferably 20 to 70%, more preferably 22 to 55%, even more preferably from 25 to 52%, e.g. 35 to 52% by weight, based on the total weight of the composition, the total weight of the composition being, in case of a tablet, the total tablet core weight.

One or more pharmaceutically acceptable carriers or diluents may be present in the solid pharmaceutical compositions, e.g. at least one filler; at least one disintegrant; at least one glidant; at least one lubricant; and optionally, at least one binder and/or a surfactant.

Fillers according to the invention include e.g. lactose, especially lactose monohydrate, preferably lactose monohydrate (200 mesh) or lactose spray dried, microcrystalline cellulose, e.g. PH 102, PH 101, microcrystalline silicified cellulose, starch, calcium phosphate, or a saccharide, e.g. mannitol, maltodextrin or maltose, or a mixture thereof. Preferably, lactose spray dried, microcrystalline cellulose or microcrystalline silicified cellulose, more preferably lactose spray dried and microcrystalline cellulose or lactose spray dried and microcrystalline silicified cellulose is used.

The composition of the invention preferably contains from 15 to 65%, preferably 35 to 65% by weight of a filler, based on the total weight of the composition, the total weight of the composition being, in case of a tablet, the total tablet core weight. Thus, a particularly suitable solid pharmaceutical composition contains as filler (a) from 18 to 31% by weight of lactose spray dried and from 18 to 31% by weight of microcrystalline cellulose or (b) from 18 to 31% by weight of lactose spray dried and from 23 to 31% by weight of microcrystalline silicified cellulose, calcium phosphate, or a saccharide, e.g. as mentioned previously, based on the total weight of the composition, the total weight of the composition being, in case of a tablet, the total tablet core weight.

Disintegrants according to the invention include e.g. natural starches, such as maize starch, potato starch, and the like, directly compressible starches, e.g. Sta-RX 1500, modified starches, e.g. carboxymethyl starches and sodium starch glycolate, starch derivatives such as amylase, crosslinked polyvinylpyrrolidones, e.g. crospovidones, e.g. Polyplasdone® XL or Kollidon® CL, alginic acid or sodium alginate, methacrylic acid divinylbenzene copolymer salts, e.g. AMBERLITE I9 IRP-88, or cross-linked sodium carboxymethylcellulose, available as e.g. AC-DI-SOL; COMMAT; PRIMELLOSEF, PHARMACEL, EXPLOCEL, or NYMCEL ZSX. Preferably, directly compressible starches, such as Sta-RX 1500 is used.

The composition of the invention preferably contains from 5 to 15% by weight of a disintegrant, based on the total weight of the composition, the total weight of the composition being, in case of a tablet, the total tablet core weight. Thus, a particularly suitable solid pharmaceutical composition contains as disintegrant from 5 to 15% by weight of a directly compressible starch, based on the total weight of the composition, the total weight of the composition being, in case of a tablet, the total tablet core weight.

Binders according to the invention include starches, e.g. potato, wheat or corn starch; hydroxypropyl cellulose; hydroxyethyl cellulose; hydroxypropylmethyl cellulose, e.g. hydroxypropylmethyl cellulose-Type 2910 USP, hypromellose, and polyvinylpyrrolidone, e.g. POVIDONE K30 from BASF. Preferably, hydroxypropylmethyl cellulose or polyvinylpyrrolidone 30 is used.

The composition of the invention may contain from 0 to 5% by weight, preferably from 1 to 5% by weight, of a binder based on the total weight of the composition, the total weight of the composition being, in case of a tablet, the total tablet core weight. Thus, a particularly suitable solid pharmaceutical composition contains as binder (a) from 0 to 3% by weight of hydroxypropyl methyl cellulose or (b) from 0 to 5% by weight of polyvinylpyrrolidone 30, based on the total weight of the composition, the total weight of the composition being, in case of a tablet, the total tablet core weight.

The composition of the invention may contain from 0 to 3% of a surfactant based on the total weight of the composition, the total weight of the composition being, in case of a tablet, the total tablet core weight. Surfactants according to the invention include e.g. an anionic, cationic or non-ionic surfactant or a mixture thereof, e.g. sodium lauryl sulfate, cetrimide, a polysorbate or a sorbitan fatty acid ester, e.g. a sorbitan fatty acid ester from a fatty acid such as oleic, stearic or palmitic acid.

Glidants according to the invention include e.g. silica, colloidal silica, e.g. colloidal silicon dioxide, e.g. AEROSIL 200, magnesium trisilicate, powdered cellulose, starch and talc. Preferably, colloidal silicon dioxide is used.

The composition of the invention preferably contains from 0.5 to 1% by weight of a glidant, based on the total weight of the composition, the total weight of the composition being, in case of a tablet, the total tablet core weight. Thus, a particularly suitable solid pharmaceutical composition contains as glidant from 0.5 to 1% by weight of colloidal silicone dioxide, based on the total weight of the composition, the total weight of the composition being, in case of a tablet, the total tablet core weight.

Lubricants according to the invention include e.g. Mg-, Al- or Ca-stearate, PEG 4000-8000, talc, sodium benzoate, glyceryl mono fatty acid, e.g. having a molecular weight of from 200 to 800 Daltons, e.g. glyceryl monostearate (e.g. Danisco, UK), glyceryl dibehenate (e.g. COMPRITOLAT™ 0888, Gattefossé France), glyceryl palmito-stearic ester (e.g. PRECIROL™, Gattefossé France), polyoxyethylene glycol (PEG, BASF), hydrogenated cotton seed oil (Lubitrab, Edward Mendell Co Inc) and castor seed oil (Cutina HR, Henkel). Preferably, magnesium stearate is used.

The composition of the invention preferably contains from 0.5 to 2% by weight of a lubricant, based on the total weight of the composition, the total weight of the composition being, in case of a tablet, the total tablet core weight. Thus, a particularly suitable solid pharmaceutical composition contains as lubricant from 0.5 to 2% by weight of magnesium stearate, based on the total weight of the composition, the total weight of the composition being, in case of a tablet, the total tablet core weight.

The composition of the invention may be in the form of a powder, granule or pellets or a unit dosage form, for example a tablet or capsule. Preferably, the solid pharmaceutical composition of the invention is in the form of a tablet. The composition of the present invention is well-adapted for direct compression into tablets. The tablets may optionally be coated, for instance with a coating comprising, a polysaccharide, e.g. cellulose, hydroxypropyl-methylcellulose, e.g. HMPC 603, polyoxyethylene glycol, e.g. PEG 6000 or PEG 8000, one or more dyers, carnauba wax, or an aluminium lake.

The composition of the invention may show good stability characteristics as indicated by standard stability trials, for example having a shelf life stability of up to one, two or three years, and even longer. Stability characteristics may be determined, e.g. by measuring decomposition products by HPLC analysis after storage for particular time periods, at various temperatures, e.g. 20°, 40° or 60° C.

The composition of the present invention may be produced by standard processes, for instance by conventional mixing, compacting, granulating, compression, or coating with or without sugar. Procedures which may be used are known in the art, e.g. those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed. (Mack Publ., Co., 1970) or later editions.

In one aspect, the present invention relates to a process for producing a composition of the invention, comprising: (a) mixing a water sensitive drug, preferably an indolylmaleimide derivative with a filler, a disintegrant, a glidant and, optionally, a binder; (b) milling and/or granulating or compacting the mixture obtained in (a); and (c) mixing the milled and/or granulated mixture obtained in (b) with a lubricant.

By using this process, a preparation having a good level of content and blend uniformity (i.e. a substantially uniform distribution of the a water sensitive drug, preferably an indolylmaleimide derivative throughout the composition), dissolution time and stability is obtained.

The process may be carried out by dry mixing the components. In this embodiment the milling step (b) may suitably comprise passing the mixture obtained in (a) through a screen, which preferably has a mesh size of 900 to 1000 μm.

The lubricant, e.g. magnesium stearate, is preferably pre-screened, e.g. with a 800 to 900 μm screen, before mixing.

Alternatively, a wet granulation process may be employed. In this embodiment, the drug is preferably first dry-mixed with the further components of the composition. Water or a granulation liquid is then added and the mixture granulated, e.g. using an automated granulator. The granules are then dried and milled.

The composition of the tablet, e.g. the tablets or capsules, may be coloured or marked so as to impart an individual appearance and to make them instantly recognizable. The use of dyes can serve to enhance the appearance as well as to identify the forms. Dyes suitable for use in pharmacy typically include e.g. carotinoids, iron oxides, chlorophyll, titanium dioxides or aluminium lakes.

The composition of the invention may be used for the treatment or prevention of the diseases for which the active agent it contains, is useful. Thus, the composition of the invention comprising an indolylmaleimide derivative of formula I may be used in the treatment and/or prevention of diseases or disorders mediated by T lymphocytes and/or PKC, e.g. acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock. The compounds of formula I are also useful in the treatment and/or prevention of T-cell mediated acute or chronic inflammatory diseases or disorders or autoimmune diseases, e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

For the above uses the required dosage will of course vary depending on the particular condition to be treated and the effect desired. In general, satisfactory results are indicated to be obtained systemically at daily dosages of from about 0.1 to about 100 mg/kg body weight. An indicated daily dosage in the larger mammal, e.g. humans, is in the range from about 0.5 mg to about 2000 mg, conveniently administered, for example, in divided doses up to four times a day.

The composition of the invention may be administered in conjunction with a co-agent depending on the diseases or disorders to be treated and the active agent present in the composition. The composition of the invention comprising an indolylmaleimide derivative of formula I may be administered in conjunction, either simultaneously or in sequence, with other drugs in immunomodulating regimens or other anti-inflammatory agents e.g. for the treatment or prevention of allo- or xenograft acute or chronic rejection or inflammatory or autoimmune disorders. For example, they may be used in combination with cyclosporines, or ascomycines or their immunosuppressive analogs or derivatives, e.g. cyclosporin A, cyclosporin G, FK-506, ABT-281, ASM 981; an mTOR inhibitor, e.g. rapamycin, 40-O-(2-hydroxy-ethyl)-rapamycin, CC1779, ABT578, biolimus-7, biolimus-9, TAFA-93, AP23573, AP23464 or AP23841 etc.; corticosteroids; cyclophosphamide; azathioprene; methotrexate; a S1P receptor modulator, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CD3, CD4, CD 11a/CD18, CD7, CD25, CD 27, B7, CD40, CD45, CD58, CD 137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists. The composition of the invention comprising an indolylmaleimide derivative of formula I may also be administered in conjunction with, e.g. simultaneously or in sequence, an antiproliferative drug, e.g. a chemotherapeutic drug, e.g. in cancer treatment, or with an anti-diabetic drug in diabetes therapy. Dosages of the co-administered immunosuppressant, immunomodulatory, anti-inflammatory, antiproliferative or anti-diabetic agent may vary depending on the type of the co-agent used, on the specific drug employed, on the condition being treated and so forth.

In accordance with the foregoing the present invention further provides:
1.1 A solid pharmaceutical composition suitable for oral administration, as defined above, for use in preventing or treating disorders or diseases mediated by T lymphocytes and/or PKC, e.g. such as indicated above, in a subject in need of such treatment.
2.1 A method for preventing or treating disorders or diseases mediated by T lymphocytes and/or PKC, e.g. such as indicated above, in a subject in need of such treatment, which method comprises administering to said subject an effective amount of a solid pharmaceutical composition suitable for oral administration as defined above.
2.2 A method for preventing or treating acute or chronic transplant rejection or T-cell mediated inflammatory or autoimmune diseases, e.g. as indicated above, in a subject in need of such treatment, which method comprises administering to said subject an effective amount of a solid pharmaceutical composition suitable for oral administration as defined above.
2.3 A method for preventing or treating disorders or diseases mediated by T lymphocytes and/or PKC, e.g. such as indicated above, in a subject in need of such treatment, which method comprises co-administration, e.g. concomitantly or in sequence, of a therapeutically effective amount of a solid pharmaceutical composition suitable for oral administration as defined above, and a second drug substance, said second drug substance being an immunosuppressant, immunomodulatory, anti-inflammatory, antiproliferative or anti-diabetic drug, e.g. as indicated above.
3. A therapeutic combination, e.g. a kit, comprising a) a solid pharmaceutical composition suitable for oral administration as defined above, and b) at least one second agent selected from an immunosuppressant, immunomodulatory, anti-inflammatory, antiproliferative and anti-diabetic drug. Component a) and component b) may be used concomitantly or in sequence. The kit may comprise instructions for its administration.
4. Use of a solid pharmaceutical composition suitable for oral administration as defined above for the preparation of a medicament for the prevention or treatment of disorders or diseases mediated by T lymphocytes and/or PKC, e.g. such as indicated above.

The invention will now be described with reference to the following specific embodiments.

The following Examples are illustrative of the instant invention, without limitating.

Compound A: 3-(1.H.-indol-3-yl)-4-[2-(4-methyl-piperazin-1-yl)-quinazolin-4-yl]-pyrrole-2,5-dione acetate salt

Sta-RX 1500: directly compressible starch from Colorcon.

### EXAMPLE 1

250 g of Compound A is mixed with 200 g lactose spray dried, 200 g microcrystalline cellulose, 12.5 g hydroxypropyl methyl cellulose 3 cps, 40 g Star-RX 1500 and 2.5 g colloidal silicon dioxide (Aerosil 200) and subsequently screened. The mixture is then milled in a Frewitt MGI device (Key International Inc. USA) using a 1000 μm mesh screen. Magnesium stearate is screened using a 800 μm mesh screen and 5 g of the screened magnesium stearate is blended with the Compound A mixture to produce a product composition. The product composition is then compacted on a tablet press using a 18 mm long die to form 250 mg strength tablets, each containing: 250 mg Compound A, 200 mg lactose spray dried, 200 mg cellulose microcrystalline, 12.5 mg hydroxypropyl methyl cellulose 3 cps, 40 mg Star-RX 1500 and 2.5 mg colloidal silicon dioxide, 5 mg magnesium stearate. Finally, a conventional water-based film coat is applied.

### EXAMPLE 2

In a further example, the process of example 1 is repeated except that the microcrystalline cellulose is replaced by microcrystalline silicified cellulose.

### EXAMPLE 3

In a further example, the process of example 1 is repeated except that the hydroxypropylmethyl cellulose is replaced by polyvinylpyrrolidone 30.

### EXAMPLE 4

The procedure of Examples 1 to 3 is repeated, except that Compound A is replaced by 3-(1.H.-indol-3-yl)-4-[2-(piperazin-1-yl)-quinazolin-4-yl]-pyrrole-2,5-dione.

### EXAMPLE 5

The procedure of Examples 1 to 3 is repeated, except that Compound A is replaced by 3-[3-(4,7-diaza-spiro[2.5]oct-7-yl)-isoquinolin-1-yl]-4-(7-methyl-1H-indol-3-yl)-pyrrole-2,5-dione.

1. A solid pharmaceutical composition suitable for oral administration comprising an indolylmaleimide derivative of formula I 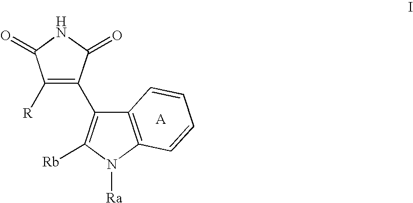
wherein
Rₐ is H; C₁₋₄alkyl; or C₁₋₄alkyl substituted by OH, NH₂, NHC₁₋₄alkyl or N(di-C₁₋₄alkyl)₂;
R_{b} is H; or C₁₋₄alkyl;
R is a radical of formula (a), (b), (c), (d), (e) or (f) 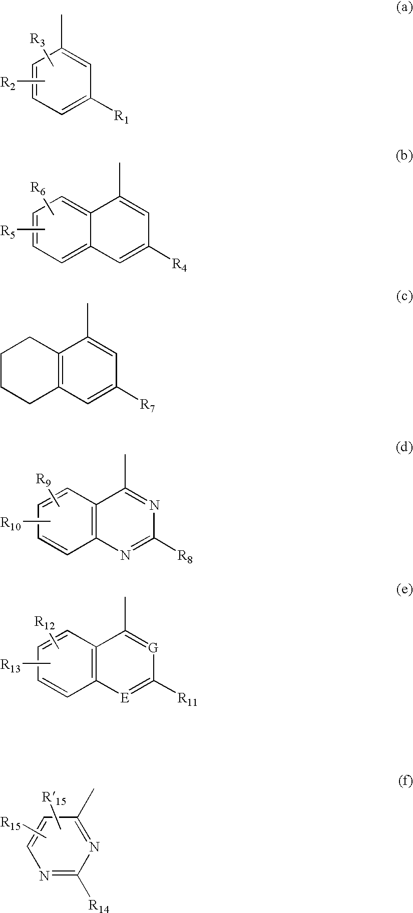
wherein
each of R₁, R₄, R₇, R₈, R₁₁ and R₁₄ is OH; SH; a heterocyclic residue; NR₁₆R₁₇ wherein each of R₁₆ and R₁₇, independently, is H or C₁₋₄alkyl or R₁₆ and R₁₇ form together with the nitrogen atom to which they are bound a heterocyclic residue; or a radical of formula α
—X—R_{c}—Y (α)
wherein X is a direct bond, O, S or NR₁₈ wherein R₁₈ is H or C₁₋₄alkyl,
R_{c} is C₁₋₄alkylene or C₁₋₄alkylene wherein one CH₂ is replaced by CRₓR_{y} wherein one of Rₓ and R_{y} is H and the other is CH₃, each of Rₓ and R_{y} is CH₃ or Rₓ and R_{y} form together —CH₂—CH₂—, and
Y is bound to the terminal carbon atom and is selected from OH, a heterocyclic residue and —NR₁₉R₂₀ wherein each of R₁₉ and R₂₀ independently is H, C₃₋₆cycloalkyl, C₃₋₆cycloalkyl-C₁₋₄alkyl, aryl-C₁₋₄alkyl or C₁₋₄alkyl optionally substituted on the terminal carbon atom by OH, or R₁₉ and R₂₀ form together with the nitrogen atom to which they are bound a heterocyclic residue;
each of R₂, R₃, R₅, R₆, R₉, R₁₀, R₁₂, R₁₃, R₁₅ and R′₁₅, independently, is H, halogen, C₁₋₄alkyl, CF₃, OH, SH, NH₂, C₁₋₄alkoxy, C₁₋₄alkylthio, NHC₁₋₄alkyl, N(di-C₁₋₄alkyl)₂ or CN;
either E is —N═ and G is —CH═ or E is —CH═ and G is —N═; and
in free form or in pharmaceutically acceptable salt.

2. A composition according to claim 1 wherein the composition comprises 20 to 70% by weight of the indolylmaleimide derivative, based on the total weight of the composition, the total weight of the composition being, in case of a tablet, the total tablet core weight.

3. A composition according to claim 1 comprising in addition at least one filler.

4. A composition according to claim 3 wherein the composition comprises from 15 to 65% by weight of the filler, based on the total weight of the composition, the total weight of the composition being, in case of a tablet, the total tablet core weight.

5. A composition according to claim 1 comprising at least one disintegrant.

6. A composition according to claim 5 wherein the composition comprises from 5 to 15% by weight of the disintegrant, based on the total weight of the composition, the total weight of the composition being, in case of a tablet, the total tablet core weight.

7. A composition according to claim 1 comprising at least one glidant.

8. A composition according to claim 7 wherein the composition comprises from 0.5 to 1% by weight of the glidant, based on the total weight of the composition, the total weight of the composition being, in case of a tablet, the total tablet core weight.

9. A composition according to claim 1 comprising at least one lubricant.

10. A composition according to claim 9 wherein the composition comprises from 0.5 to 2% by weight of the lubricant, based on the total weight of the composition, the total weight of the composition being, in case of a tablet, the total tablet core weight.

11. A composition according to claim 1 comprising at least one binder.

12. A composition according to claim 11 wherein the composition comprises from 0 to 5% by weight of the binder, based on the total weight of the composition, the total weight of the composition being, in case of a tablet, the total tablet core weight.

13. A composition according to claim 1 comprising at least one surfactant.

14. A composition according to claim 13 wherein the composition comprises from 0 to 3% by weight of the surfactant, based on the total weight of the composition, the total weight of the composition being, in case of a tablet, the total tablet core weight.

15. A composition according to claim 3 wherein the filler is selected from lactose, microcrystalline cellulose, microcrystalline silicified cellulose, starch, calcium phosphate and saccharide.

16. A composition according to claim 5 wherein the disintegrant is selected from natural starches, directly compressible starches, modified starches, starch derivatives, crosslinked polyvinylpyrrolidones, alginic acid or sodium alginate, methacrylic acid divinylbenzene copolymer salts and cross-linked sodium carboxymethylcellulose.

17. A composition according to claim 7 wherein the glidant is selected from silica, colloidal silica, magnesium trisilicate, powdered cellulose, starch and talc.

18. A composition according to claim 9 comprising magnesium stearate as lubricant.

19. A composition according to claim 11 wherein the binder is selected from starches, hydroxypropyl cellulose, hydroxyethyl cellulose, hydroxypropylmethyl cellulose, hypromellose and polyvinylpyrrolidone.

20. A composition according to claim 1 wherein the composition is in the form of a capsule or a tablet, the tablet being optionally coated.

21. A composition according to claim 1, wherein the indolylmaleimide derivative comprises 3-(1.H.-indol-3-yl)-4-[2-(4-methyl-piperazin-1-yl)-quinazolin-4-yl]-pyrrole-2,5-dione or a pharmaceutically acceptable salt thereof.

22. A composition according to claim 1, wherein the indolylmaleimide derivative comprises 3-(1.H.-indol-3-yl)-4-[2-(piperazin-1-yl)-quinazolin-4-yl]-pyrrole-2,5-dione or a pharmaceutically acceptable salt thereof.

23. A composition according to claim 1, wherein the indolylmaleimide derivative comprises 3-[3-(4,7-diaza-spiro[2.5]oct-7-yl)-isoquinolin-1-yl]-4-(7-methyl-1H-indol-3-yl)-pyrrole-2,5-dione or a pharmaceutically acceptable salt thereof.

24. A composition according to claim 1 for use in preventing or treating disorders or diseases mediated by T lymphocytes and/or PKC in a subject in need of such treatment.

25. A process for producing a solid pharmaceutical composition suitable for oral administration according to claim 7 comprising: (a) mixing an indolylmaleimide derivative of formula I as defined in claim 1 with a filler, a disintegrant, a glidant and, optionally, a binder; (b) mixing, dry compacting, milling, granulating, drying or compacting the mixture obtained in (a); (c) mixing the mixture obtained in (b) with a lubricant; (d) optionally tableting and (e) optionally coating.

26. A process according to claim 25 wherein in step (b) the mixture is wet granulated, roller compacted or compressed.

27. A method for preventing or treating disorders or diseases mediated by T lymphocytes and/or PKC in a subject in need of such treatment, which method comprises administering to said subject an effective amount of a solid pharmaceutical composition suitable for oral administration according to claim 1.

28. (canceled)
